(19) United States
(12) Patent Application Publication
(10) Pub. No.: US 2014/0287449 A1
(43) Pub. Date: Sep. 25, 2014

(54) Photosynthetic microorganism condition detection sensor

(71) Applicant: David Bonyuet, Watertown, MA (US)

(72) Inventor: David Bonyuet, Watertown, MA (US)

(73) Assignee: 4-oem, llc, Watertown, MA (US)

(21) Appl. No.: 13/998,976

(22) Filed: Dec. 30, 2013

Related U.S. Application Data
(60) Provisional application No. 61/848,230, filed on Dec. 28, 2012.

(51) Int. Cl. C12Q 1/02

(52) U.S. Cl. CPC C12Q 1/02
USPC 435/29; 435/288.7

(57) ABSTRACT

The present invention provides an optical probe apparatus and method for microorganism culture monitoring. The optical probe can be immersed within the microorganisms and include at least one emitter and at least two detectors that excite photosynthetic pigments in the culture medium. The optical probe can measure the culture spectral characteristics, targeting those that are an indication of the healthiness and productivity condition. The optical probe can also include a microcontroller and storage. The microcontroller can compare past measurements of the optical probe with current measurements and determine a health status of the microorganisms. The optical probe is optimized to measure spectral characteristics from the microorganism in real time. The present invention relates to a sensor tune to detect the healthiness condition of photosynthetic microorganisms.

### RELATED APPLICATION

This application claims benefit of U.S. Provisional Patent Application No. 61/848,230 filed on Dec. 28, 2012, the disclosures of which are hereby incorporated herein by reference in their entireties.

### FIELD OF THE INVENTION

This invention provides a sensor apparatus and method capable of providing early indication of the photosynthetic microorganisms healthiness condition, degradation and productivity status. In other embodiments the sensor apparatus and method is capable of determining the existence of small amounts of photosynthetic organisms in the process flow, disregarding any other particles in the same liquid. In certain embodiments the invention is capable of working as a standalone system to provide easy information to respond in a proactive manner to field issues related to photosynthetic cultures that might require immediate attention due to a surge in temperature, contamination, food depletion, etc; some of these actions might imply release of antibiotics in the culture, coolants in the system, nutrients in the media, vitamin addition, etc. In a different context this sensor can provide early indications of small trace amounts of photosynthetic organisms in the flow despite the presence of other particles or liquid turbidity.

### BACKGROUND OF THE INVENTION

Photosynthetic organisms can grow unintentionally as part of the nature or can be harvested in specialized ponds or bioreactors. In the first case, their presence is not desired, therefore they must be detected in order to trigger sanitation and disinfectants processes. In the second case they are desired and it is important to evaluate their correct growth from the very moment of the inoculation itself. Photosynthetic culture (algae, bacteria, etc) for industrial processes is grown in water enriched with additional nutrients (typically called media). This photosynthetic culture is characterized by a green color; from the moment of inoculation there is a very faint green shade and as more culture growth the greenness becomes darker and darker. The shade of green are determine basically by the type of media and the type of culture. In bioreactor processes (close processes) the nature of the organism is usually very specific (only one type of organism is used). The traditional way to measure the photosynthetic organic culture growth is by determining the “darkness” of the liquid, and the reason of this is because the amount of cells increase over time and with more organisms in the same volume the less amount of light can pass through it.

In some occasions, the photosynthetic culture does not have the right greenness and this effect is called chlorosis, which is a condition in which photosynthetic organisms have degraded photosynthetic pigments. As photosynthetic pigments are responsible for the green shade color in photosynthetic organisms, culture with chlorosis tend to be yellowish, brownish in color. There are multiple factors that can affect photosynthetic cultures and deviate them into chlorosis, among them photo inhibition, excessive heat, lack of nutrients, inadequate cooling, etc. Chlorosis can affect photosynthetic culture's quality, productivity and or their by-products, whether that is the biomass or additional chemicals in the process. Chlorosis is also a way to determine the effectivity of disinfectants in the treatment of liquid (including water supplies).

There are several ways to measure the growth of any organic culture, most of them in the laboratory. The most wide spread method is through turbidimeters as for example the one described by patents U.S. Pat. No. 2,964,640 to Wippler, U.S. Pat. No. 2,892,378 to Canada, U.S. Pat. No. 4,152,070 to Moore, all of which are incorporated herein by reference. Turbidimeters measure the clarity of liquids by measuring the amount of light that can pass through it. Particles suspended in a fluid will obstruct the amount of light that can traverse the liquid and therefore the more particles the higher the turbidity. This method however, does not take into account the photosynthetic nature of the culture which tend to be more sensible in the photosynthetically active region (PAR) more than in other regions. Furthermore different cultures have different absorption pattern and therefore with a similar cell density they might present different turbidity readings. Furthermore, turbidimeters are not tune to take into account the effect of chlorosis and it does not provide any indication on photosynthetic culture healthiness or productivity.

Another alternative to measure culture growth is by using scientific instrumentation known as spectrometers as for example the one described in patents U.S. Pat. No. 7,196,790 by Cole, U.S. Pat. No. 8,529,218 by Makarov, U.S. Pat. No. 8,502,981 by Bonyuet, all of which are incorporated herein by reference. High-performance liquid chromatography (HPLC) is a technique in analytic chemistry used to separate the components in a mixture, to identify and quantify each component also used to quantify and qualify photosynthetic organisms; this is mostly an off-line destructive method used to separate chlorophylls and carotenoids by pumping a mobile phase through a densely packed column; after the separation, the detection is performed by a photodiode array (PDA) detector that scans the absorbance of the eluent at a range of wavelengths as for example in patents U.S. Pat. No. 4,656,141 by Birks, U.S. Pat. No. 5,240,577 by Jorgenson. all of which are incorporated herein by reference. Ultraviolet-visible spectroscopy or ultraviolet-visible spectrophotometry (UV-Vis or UV/Vis) refers to absorption spectroscopy or reflectance spectroscopy in the ultraviolet-visible spectral region which can be used for photosynthetic organism analysis as described by patents: U.S. Pat. No. 6,559,941 by Hammer, U.S. Pat. No. 7,196,790 by Cole, all of which are incorporated herein by reference. All these instrumentation are capable of sweeping a range of wavelength and therefore provide better information on the compound to be analyzed. These instrumentation are not designed to analyze photosynthetic culture. Furthermore, the user need to tune and understand what portions of the wavelength are required to be analyzed. In addition, the relationship associated with all the different compounds are not taken into account by these equipment.

Fluorescence spectroscopy (also known as fluorometry or spectrofluorometry) is a type of electromagnetic spectroscopy which analyzes fluorescence response from a sample. It involves using a beam of light, usually ultraviolet light, that excites the electrons in molecules of certain compounds and causes them to emit light; typically, but not necessarily, visible light as described for example in patents U.S. Pat. No. 3,604,927 by Hirschfeld, U.S. Pat. No. 3,947,125 by West, all of which are incorporated herein by reference. Fluorometers are devices that measures fluorescence response of compounds by supplying an excitation light source, detecting the resulting emission light at a different wavelength. U.S. Pat. No. 7,099,012 by Crawford and U.S. Pat. No. 6,369,894 by Rasimas, all of which are incorporated herein by reference. These instrumentations can measure fluorescence response of photosynthetic organisms but measuring a single response type might lead to misinterpretations and the system must be tuned to specific compounds to be detected and it requires further skills and analysis to get an accurate reading.

Another alternative is the usage of particle concentrations sensors as for example the one described by patents: U.S. Pat. No. 8,327,692 by Cho, US 20,130,248,453 by Allier, U.S. Pat. No. 8,405,033 by Debreczeny, all of which are incorporated herein by reference. These sensors count the concentration of particles in the fluid but does not evaluate the photosynthetic culture in terms of healthiness nor productivity. Chlorophyll meters are also an alternative method to measure photosynthetic cultures as in U.S. Pat. No. 4,295,042 by Kuzunuki and WO2013151862 by Greenbaum, both of which are incorporated herein by reference, but they fail to measure other critical photosynthetic pigments that are responsible for the chlorosis; these methods and devices are not adequate for the bioreactors in on line processes. Furthermore some of these solutions are laboratory base and or sample base which is a significant inconvenient for large scale bioreactors for the cultivation of photosynthetic culture at mass scale.

### BACKGROUND OF THE INVENTION

As described in the background of the invention there are previous systems and methods that provide some basic data but cannot provide direct information about the culture condition either in term of healthiness or productivity without intensive analysis and expert evaluation. Furthermore existing methods do not provide real time historical data (or data trending) that would inform about the successful growth or effectivity of disinfectants. Organic culture degradation (i.e. chlorosis, etc.) is a significant issue that requires early intervention to prevent the loss of significant process volume and their by-products, not to mention the difficulties associated to a process reinitialization: draining, inactivation, cleaning, disinfection, Clean-in-place (CIP), Steam-in-Place (SIP), media injection, inoculation, water addition, nutrient addition, ozone injection, etc. On the other hand, inadequate detection of the efficiency of disinfectants on water treatment can lead to inadequate or hazardous condition of water supplies or expensive over-treatment. Therefore, a sensor apparatus and method capable of providing early indication of the photosynthetic microorganisms healthiness condition, degradation and productivity status is of significant interest. It is also of importance a sensor capable of determining the existence of small amounts of photosynthetic organisms in the process flow, disregarding any other particles in the same liquid.

The present invention addresses the needs described above and it is intended to work as a standalone system to provide early and easy information to respond in a proactive manner to field issues related to photosynthetic cultures that might require immediate attention due to a surge in temperature, contamination, nutrient depletion, etc. Some of these actions might imply release of antibiotics in the culture, coolants in the system, nutrients in the media, etc. Embodiments of the present invention provide a probe, system, or method thereof. The optical probe can be immersive within the microorganisms and culture medium and include at least one emitter and at least two detectors.

There are applications were photosynthetic microorganisms are used for commercial process manufacturing, like nutrients, amino acids, antibiotics, vitamins, food, fuels, biofuels, biomass or other substances etc. This is especially because those organisms are capable of synthesizing those chemicals internally or becoming nutraceutical products, pharmacological products, industrial derivatives, biomass, chemical compounds, etc.) Those microorganisms could be bacteria, algae, microalgae and others that use solar radiation and the photosynthetic process to perform in part or in whole the conversion required. These microorganisms will be highly productive when they are healthy (well fed with nutrients, not photo-inhibited, etc) and not impacted by stressful conditions (excessive heat, etc). This sensor includes a series of optical detectors that excite the forward, reflectance and fluorescence features on the organisms and through further calculations determine their healthiness conditions. The present device, system and methods address this need.

Optical density is a way to measure the amount of light that can pass through a material, in some cases, turbidity is used as a related terminology. Presently some turbidity detectors are designed to determine the “cloudiness” of water (the presence of particles in the water that can affect the light passing through it). Others require a reference source sample or reference detector to operate. Other detectors require a substantial amount of power to operate in a continuous manner. Other detectors require electrodes and alternate current passing through the culture and are designed to be laboratory equipments. Others cannot measure the optical density from low density all the way to full cloudiness without a dilution processes. Others are not specifically targeted to measure organic photosynthetic culture. Some other methods are based on sampling techniques.

What is needed, therefore, is a device, system, or method that takes into account the nature of biological culture, their unique photosynthetic capability and how the organism's growth affects the optical density and measures the parameters that are relevant to organism performance and its healthiness condition. A probe and system to be used in-line and/or on-line of commercial scale processes in continuous operation mode or in batch operation mode is needed to facilitate field operations. An optical probe that senses multiple wavelengths of emission, including reflexion and fluorescence would be advantageous because it will enable the determination of the culture health condition and the optical density for photosynthetic organisms. An optical probe that can measure the measure the response of the microorganisms to the light of a wavelength that is photosynthetically active in the phototrophic microorganism will be able to provide information on the culture performance. A probe and system providing integrated sensing capabilities that is scalable, low cost, and efficient for measuring organism's growth and or health. The culture sensor system may need to have low material cost, provide for easy deployment, minimal or no maintenance and minimal or no calibration. A probe and system that can indicate the organism health condition by monitoring the microorganism culture optical photosynthetic response is necessary.

### SUMMARY OF THE INVENTION

This invention provides a sensor apparatus and method capable of providing early indication of the photosynthetic microorganisms healthiness condition, degradation and productivity status. In other embodiments the sensor apparatus and method is capable of determining the existence of small amounts of photosynthetic organisms in the process flow, disregarding any other particles in the same liquid. In certain embodiments the invention is capable of working as a standalone system to provide easy information to respond in a proactive manner to field issues related to photosynthetic cultures that might require immediate attention due to a surge in temperature, contamination, food depletion, etc; some of these actions might imply release of antibiotics in the culture, coolants in the system, nutrients in the media, vitamin addition, etc. In a different context this sensor can provide early indications of small trace amounts of photosynthetic organisms in the flow despite the presence of other particles or liquid turbidity.

Other embodiments include one or more of the following variations. The probe can also include a microcontroller and storage. The microcontroller can compare past measurements of the optical probe with current measurements and determines a health status of the microorganisms. The at least one emitter and the at least two detector can be modulated. The at least one emitter can have wavelengths equal or greater than 360 nm. The at least one emitter can have wavelengths equal to or lower than 1,200 nm. The optical probe can include a first detector located at a 90 degree angle from a focused beam of light emitted from the emitter and a second detector located in line with the focused beam of light. The optical probe can include a first detector located at a 45 degree angle from a focused beam of light emitted from the emitter and a second detector located in line with the focused beam of light. The optical probe can include a first detector located at a 10 degree angle from a focused beam of light emitted from the emitter and a second detector located in line with the focused beam of light. The optical probe can include a first detector located at the same location as the emitter for detecting reflected light. The optical probe can measure an optical density of the microorganisms by comparing the incident light to the reflected or scattered or transmitted light. A microcontroller and storage can compare past measurements of the optical probe with current measurements. The microcontroller and storage can compare past measurements of the optical probe with current measurements and determine a health status of the microorganisms. An ambient light detector, a microcontroller and storage can be used to compare past measurements of the optical probe with current measurements. An immersive optical probe can operate in a continuous mode or in discrete mode. The optical probe can measure an optical density of the microorganisms by emission and detection at a wide range of wavelength, from 360 nm to 1,200 nm. The optical probe can measure the optical density of the microorganisms by emission and detection at specific wavelengths: 440 nm, 500 nm, 630 nm, 640 nm, 680 nm, 730 nm, 880 nm, and 1000 nm. The optical probe performs calculations based on the optical measurement at different wavelengths to determine the culture health and or nutrients conditions in the culture. The optical probe uses multiple wavelengths to determine the optical density of the culture to overcome the potential presence of other compounds in the fluid (contaminants, dirt, etc).

The optical probe can measure a color of reflected light off the microorganisms. The optical probe can measure a color of reflected light off the microorganisms by reflection and detection at a wavelengths ranging from 560 to 640 nm. The optical probe can measure a color of reflected light off the microorganisms by reflection and detection at a wavelength of about 440 nm, 560 nm, 580 nm, 590 nm, 600 nm and 640 nm. A microcontroller and storage can be used to measure a color of reflected light off the microorganisms by emission and detection at a wavelength of about 440 nm, 560, 580 nm, 590 nm, 600 nm and 630 nm. The microcontroller can compare past color measurements of the optical probe with current color measurements and determine a health condition status of the microorganisms.

In another embodiment the optical probe uses multiple wavelengths to measure absorption, fluorescence and scattered light from the culture. Multiple emitters can be multiplexed to operate at different intervals and times. Alternatively, multiple emitters can be used simultaneously in special sensor geometry to prevent light interference from one another. This optical probe can use absorbance, fluorescence and reflectance techniques together or separately to measure different microorganism properties and validate data collected. By measuring optical properties through this combine techniques (absorption, fluorescence and scattered light) the optical probe can have a wider dynamic range (measuring from the moment of inoculation up to higher culture densities) and minimize the effect of other elements in the organism's fluid (suspended particles, contamination, etc)

In another embodiment, the optical probe can measure the photosynthetic response of the microorganisms. The optical probe can measure a photosynthetic response of the microorganisms by emission and detection at wavelengths in the vicinity of 440 nm, 630 nm, and 640 nm. The optical probe can measure a photosynthetic response of the microorganisms by emission and detection at a wavelength of about 440 nm, 500 nm, 560 nm, 630 nm, 640 nm, and 680 nm. A processor and storage can determine the photosynthetic response by comparing past measurements of the optical probe at a wavelength of about 440 nm and 630 nm with current measurements. The optical probe can measure a photosynthetic response of the microorganisms by measuring the fluorescence of the microorganisms. The optical probe can measure a fluorescence response of the microorganisms by excitation at a wavelength of between 380 nm to 430 nm and emission detection at a wavelength of about 660 nm and 680 nm. The optical probe can measure a fluorescence response of the microorganisms by excitation at a wavelength between 560 nm and 590 nm and emission at a wavelength of 660 nm. The optical probe can measure a photosynthetic activity of the microorganisms by emission at a wavelength between 560 nm and 590 nm and detection of emission at a wavelength of about 660 nm in a portion of the reactor that is shielded from light external to the emitter.

In yet another embodiment, a method of measuring in a continuous manner photosynthetic organisms with an optical probe having at least one emitter and at least two detectors.

In yet another embodiment, a method to trigger a local switch depending on the conditions measured by the optical probe based on any of the mechanisms described above.

The present invention is not intended to be limited to a system or method that must satisfy one or more of any stated objects or features of the invention. It is also important to note that the present invention is not limited to the exemplary or primary embodiments described herein. Modifications and substitutions by one of ordinary skill in the art are considered to be within the scope of the present invention.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a basic block diagram description of this invention apparatus according to an illustrative embodiment of the present invention.

FIG. 2 is a profile block diagram of a probe constructed in accordance with an exemplary embodiment of the invention.

FIG. 3A is a graph of optical density measurements of the microorganisms using 440, 500, 640 and 680 nm in accordance with an exemplary embodiment of the invention. FIG. 3B is a graph of optical density measurements of the microorganisms using 560 nm and 660 nm in accordance with an exemplary embodiment of the invention. FIG. 3C is a graph of optical density measurements of the microorganisms using 730, 880 and 1000 nm in accordance with an exemplary embodiment of the invention.

FIG. 4A is a graph of wavelength versus absorption of the culture using the 630 nm referenced to 730 nm in accordance with an exemplary embodiment of the invention. FIG. 4B is a graph of wavelength versus absorption of the culture using the 640 nm referenced to 660 nm in accordance with an exemplary embodiment of the invention. FIG. 4C is a graph of wavelength versus absorption of the culture using only 730 nm in accordance with an exemplary embodiment of the invention. FIG. 4D shows a similar performance by referencing the wavelength of interest 640 nm against the baseline at 730 nm in accordance with an exemplary embodiment of the invention. FIG. 4E is a graph of wavelength versus absorption of the culture using the 440 nm referenced to 730 nm in accordance with an exemplary embodiment of the invention. FIG. 4F is a graph of wavelength versus absorption of the second set of culture using the 440 nm referenced to 730 nm in accordance with an exemplary embodiment of the invention.

FIG. 5 is a graph of emissions of a healthy culture and a sick one with signs of chlorosis with an excitation range from 380 nm to 470 nm and monitoring the emission at 680 nm in accordance with an exemplary embodiment of the invention.

FIG. 6A is a graph of fluorescence emissions of three healthy culture at different culture concentrations subjected to temperature increase; FIG. 6B is a graph showing an alternative trend with of fluorescence and absorbance both in accordance with an exemplary embodiment of the invention.

FIG. 7A is a graph showing the reflectance at 600 nm in accordance with an exemplary embodiment of the invention and FIG. 7B is a graph showing a similar trend with the ratio of reflectance in accordance with an exemplary embodiment of the invention.

FIG. 8 is a graph showing the color at a particular wavelength increasing as the culture transition from a healthy state to a sick one with signs of chlorosis in accordance with an exemplary embodiment of the invention.

FIG. 9A is a graph of the side-scattered emission and fluorescence of the culture with an excitation at 430 nm in accordance with an exemplary embodiment of the invention. FIG. 9B is a graph of the side-scattered emission and fluorescence of the culture with an excitation at 490 nm in accordance with an exemplary embodiment of the invention. FIG. 9C is a graph of the side-scattered emission and fluorescence of the culture with an excitation at 590 respectively in accordance with an exemplary embodiment of the invention.

FIG. 10A is a block diagram of a probe with discrete components and at least two detectors in accordance with the exemplary embodiment of the invention. FIG. 10B is a block diagram of a probe with discrete components, multiple emitters and multiple detectors in accordance with the exemplary embodiment of the invention. FIG. 10C is a block diagram of a probe with discrete components, multiple emitters, multiple detectors and a multiplexer in accordance with the exemplary embodiment of the invention.

FIG. 11 is a block diagram of a probe with integrated components, multiple emitters, and multiple detectors in accordance with the exemplary embodiment of the invention. Advanced enhanced integration in microcontroller, DSP or FPGA technologies allow for more features embedded in the same sensor.

The drawings are not necessarily to scale, emphasis instead being placed upon illustrating embodiments of the present invention.

FIG. 12A is a simple representation of the emitter and receiver's configuration to measure absorbance and reflectance or fluorescence in accordance with the exemplary embodiment of the invention. FIG. 12B is a simple representation of the emitter and receiver's configuration to measure absorbance and reflectance and fluorescence in accordance with the exemplary embodiment of the invention. FIG. 12C is a simple representation of the emitter and receiver's configuration to measure absorbance, emitter intensity and reflectance or fluorescence in accordance with the exemplary embodiment of the invention. FIG. 12D is a simple representation of the emitter and receiver's configuration to measure reflectance and fluorescence in accordance with the exemplary embodiment of the invention. FIG. 12E is a simple representation of the emitter and receiver's configuration to measure emitter intensity and reflectance or fluorescence in accordance with the exemplary embodiment of the invention.

FIG. 13 shows the flowchart of the algorithm used to implement the proposed method according to an illustrative embodiment of the present invention.

FIG. 14 shows a 3D view of one of the potential implementations of the optical sensor in accordance with an exemplary embodiment of the invention.

FIG. 15 shows a potential mounting of the receivers and optical gears in accordance with an exemplary embodiment of the invention.

### DETAILED DESCRIPTION OF THE INVENTION

A description of preferred embodiments of the invention follows. The relevant teachings of all patents, published applications and references cited herein are incorporated by reference in their entirety. The following explanations of terms and methods are provided to better describe the present invention and to guide those of ordinary skill in the art in the practice of the present invention. As used herein, “comprising” means “including” and the singular forms “a” or “an” or “the” include plural references unless the context clearly dictates otherwise. For example, reference to “comprising a phototrophic microorganism” includes one or a plurality of such phototrophic microorganisms. The term “or” refers to a single element of stated alternative elements or a combination of two or more elements, unless the context clearly indicates otherwise.

It is contemplated that methods, systems, and processes described herein encompass variations and adaptations developed using information from the embodiments described herein. Headers are used herein to aid the reader and are not meant to limit the interpretation of the subject matter described.

Unless explained otherwise, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. The materials, methods, and examples are illustrative only and not intended to be limiting. Other features of the invention are apparent from the following detailed description and the claims.

Throughout the description, where systems and compositions are described as having, including, or comprising specific components, or where processes and methods are described as having, including, or comprising specific steps, it is contemplated that, additionally, there are systems and compositions of the present invention that consist essentially of, or consist of, the recited components, and that there are processes and methods of the present invention that consist essentially of, or consist of, the recited processing steps.

The mention herein of any publication, for example, in the “Background of the Invention” section, is not an admission that the publication serves as prior art with respect to any of the claims presented herein. The “Background of the Invention” section is presented for purposes of clarity and is not meant as a description of prior art with respect to any claim.

General

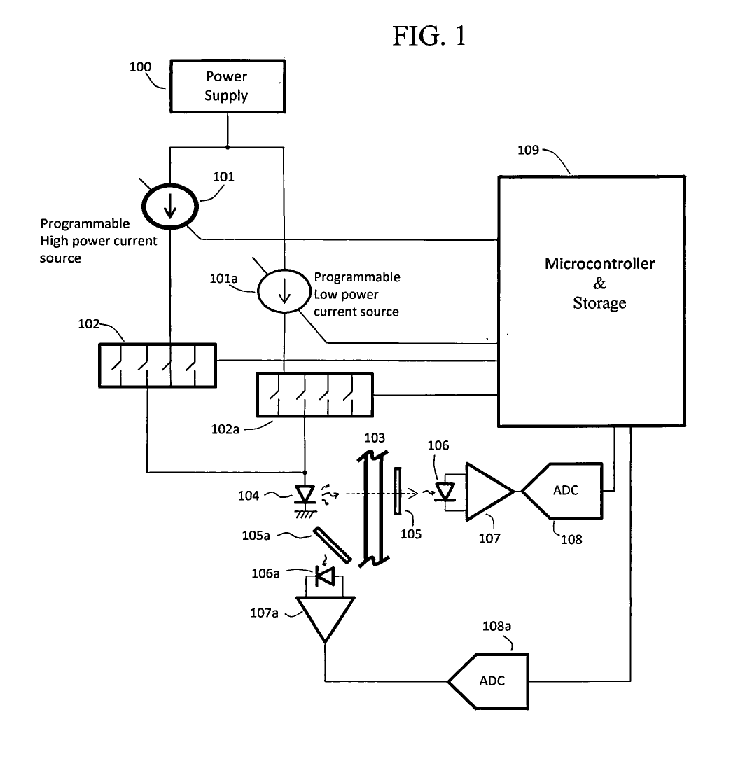

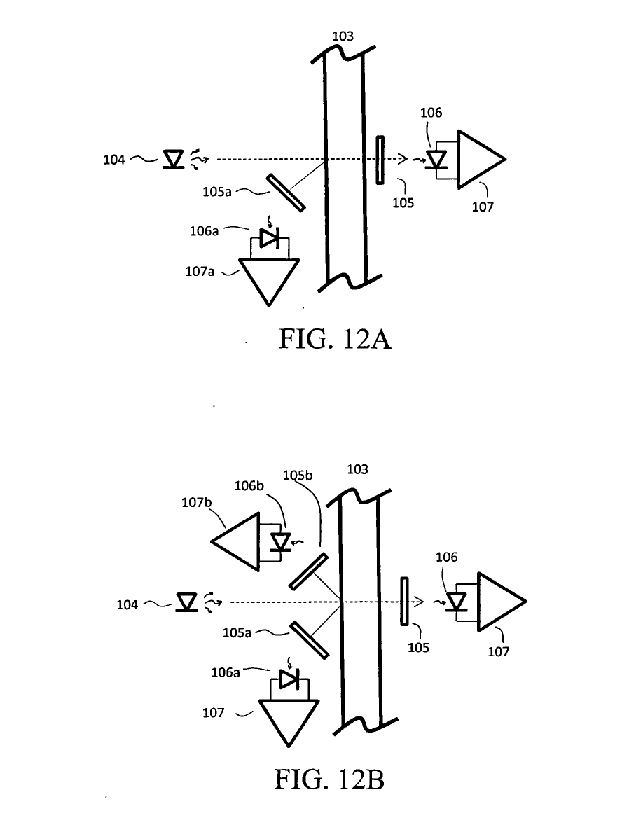

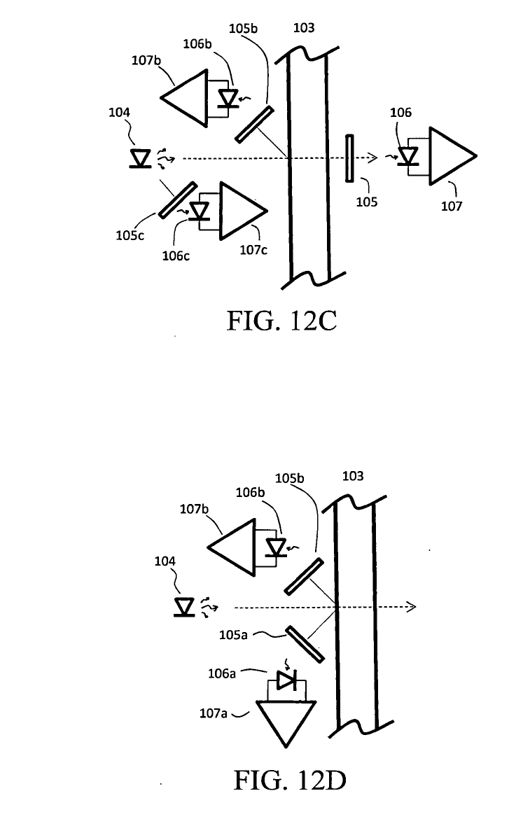

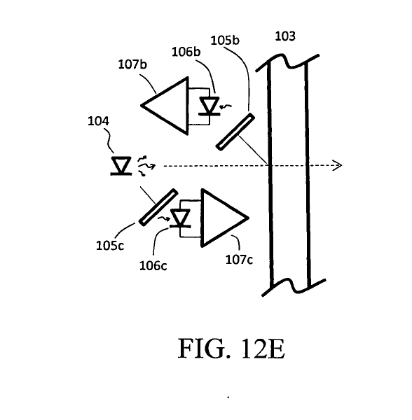

FIG. 1 is the generic block diagram of the sensor method invention to measure photosynthetic organism and determine their condition in terms of healthiness or productivity. 103 represent the aperture in the sensor for the culture to pass through. The sensor is powered through an external or internal source and the final voltage for the electronic components are derived from the power supply unit 100. A programmable low power precision current source 101a allows constant current to power low intensity emitter 104, whereas a programmable high power current source 101 is used to power emitter 104 with a higher intensity. Higher intensity is required to handle absorption measurements with culture of high turbidity and also in cases of fluorescence. The programming gain in the current sources 101 and 101a are controlled by the microcontroller. The optional analog switches 102 and 102a allows to select which emitter 104 is going to be powered: multiple emitters 104 can be connected in different configurations as shown in FIG. 12. The emitter light pass through the culture 103 and through the optional light filter 105 it is received by the receiver 106 which take the light and passed it to the transimpedance amplifier 107 which will filter and gain the signal to the ADC 108 for the microcontroller & storage 109 to process. In a similar approach, reflected light from the culture 103 is optionally filtered through 105a and detected by the receiver 106a. The ADC 108a take this analog data for the microcontroller & storage 109. There might be additional optional light blocking elements just required to block the light from interfering between the different receivers. 103 is a partial view of the aperture 208 in the sensor where the culture pass through.

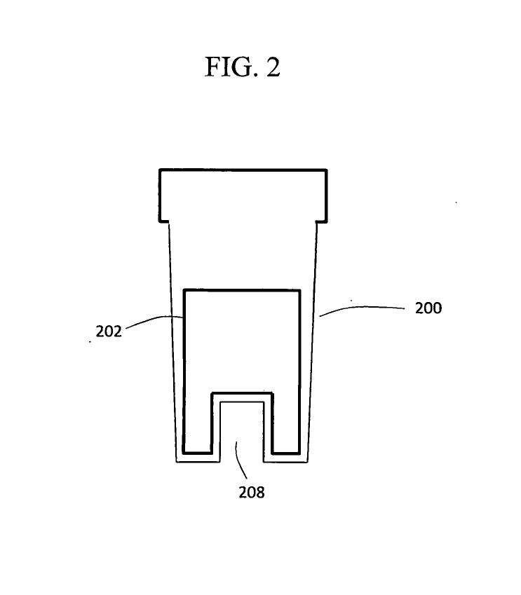

Referring to FIG. 2, an exemplary optical probe 100 200 which includes the sensor (optics, emitter, receivers, electronics, filters, holders, etc.) 202 encapsulated in the probe body 200. The probe body 200 provides an aperture 208 to facilitate the photosynthetic microorganisms' culture flow (the microorganism's fluid must pass through the aperture 208). The body of the sensor 104 200 can be made of translucent material to light of a wavelength that is photosynthetically active to the microorganisms. 103 is a partial view of the aperture 208. 202 represents the assembly of all the elements described in FIG. 1 (electronics, optics and any miscellaneous).

An optical probe 200 can be provided within the flow of to the culture medium and microorganisms in the process flow. Embodiments of the optical probe 200 can be used to take a variety of measurements to determine and/or predict the future health of the microorganisms as will be described in greater detail herein. Embodiments can provide for measurements of growing organic cultures without being affected by environmental factors, industrial noise and/or diurnal light influence. An immersive optical probe 200 can be inside the process (either on-line or in-line or inside the bioprocess vessel). Embodiments can incorporate an option to measure the color of the culture (to estimate the culture health, potentially measuring the greenness and nearby color bands, other colors might provide a good health indication too). The culture photosynthetic response can also be measured by monitoring key wavelengths associated to photosynthetic pigments. Fluorescence can also be measured to determine the photosynthetic activity. The immersive optical probe sensor 200 is designed such that the culture can pass through the aperture 208 and be immersed directly in the process flow of any closed or open bioreactor where photosynthetically organism might be present.

The probe body 200 can also include a number of devices that can support the operation of the process; for example, devices for flowing gases (e.g., carbon dioxide, air, and/or other gases), inlets and outlets, and other elements can be integrated or operationally coupled to the probe. The probe body 200 can include additional elements (not shown) such as inlets and outlets, for example, for growth media injection, pH balancing, temperature sensing, culture medium enrichment or removal, vitamin addition, antibiotic injection, etc.

The sensor 202 can include a microcontroller and electronic storage within the probe housing 200 or communicate wired or wirelessly with the microcontroller and electronic storage 109 located external to the probe housing 200. The microcontroller and storage 109 can be used to store and provide historical data that can be used to determine the microorganisms' current health or predict future health. In other embodiments, the microcontroller 109 can be provided with additional data from external sources that can provide, for example, sunlight levels, temperature, pH and/or other data from sensors in the probe.

Data received from the optical probe 200 can be used by the microcontroller 109 to determine, for example, that the system at the current temperature, pH, and lighting conditions requires additional nutrients to maintain an optimal productivity of the microorganisms. The microcontroller 109 can then activate and increase the level of nutrients supplied to the reactor. Embodiments can be used to provide an automated feedback loop. In other embodiments, the optical probe 200 and or the sensor 202 can be automated with a range of sensed values and include a range of values that activate an alarm to warn operators of undesirable conditions. The system FIG. 1 can also take into account additional data, for example, time of day or sunlight levels. In this example, the system FIG. 1 can utilize information from the optical probe 200 combined with the additional information to alter future conditions. For example, during a cloudy day approaching evening time, the system can be designed to automatically reduce nutrient load.

Optical Probe

There are multiple variables that measure the quality of cultures of microorganisms. Knowledge of these parameters in isolation, their time evolution and progress along with the relationship between them can be a factor to understanding the health and productivity of organic cultures. Among these parameters of interest for an exemplary culture can be: optical density, culture color, and photosynthetic response.

The sensor 202 can include at least one emitter 104 and at least two detectors 106 and 106a. The emitter 104 can be, for example, a laser diode or an LED as these sources are stable and have well defined relationships between current and light intensity. LEDs can require no lensing, no sophisticated temperature or current control and are therefore much simpler to implement compared to laser diodes. Furthermore, the intensity of LEDs tends to be far greater and they tend to have higher temperature stability. The detector 106 (or 106a, 106b, and 106c) can be light sensors through photocells (from, for example, Advanced Photonix®, Parallax®, or equivalent), photodetectors (such as the ones, for example, from Avago Technologies®, OSRAM Opto Semiconductors®, Hamamatsu etc.) or photodiodes (such as the ones from Vishay®, Everlight Electronics®, etc.). The photodiode can include lenses and/or filters to allow for the capture of desired wavelength light, alternatively, the photodiode can be epoxied with colored epoxy instead of light filters. Embodiments can also include a modulated emitter 104 with the detector 106 (or 106a, 106b, and 106c) tuned to the same modulation and therefore minimizing the effect of ambient and artificial light sources. The modulation can be modified in real time in both the emitter and detector to further eliminate the influence of disturbances and noise. Embodiments can also include a multiplexer 102 and 102a to switch on/off different emitters (when present); this function can be embedded in the microcontroller firmware, as will be described later herein.

The probe body 200 can be designed to hold all of the components in an encapsulated container that can be submersible within the microorganisms and culture medium during operation in the piping or process structure. The encapsulation can be designed to support CIP (clean-in-place), and it can also be designed to withstand SIP (steam-in-place) and/or autoclaving. The probe may comprise a controller 109 which can be implemented entirely through analog electronic, or with a microcontroller and supporting circuits or with a DSP (Digital Signal Processing) or with an FPGA (Field-programmable gate array) or with ASIC (application-specific integrated circuit) or any variation or combination of these components.

The probe sensor 202 as described by FIG. 1 can among other activities: (1) drive the emitter 104 with a constant current and/or a modulated current using 101 or 101a; (2) control or not the sensor and emitter temperature; (3) measure the signal from the receivers 106 (or 106a, 106b, and 106c); (4) process or measure additional variables associated to the culture (temperature, etc); (5) track time or estimation of elapsed events; (6) store data or accumulates all the variables measured or calculated at specific intervals or time. The optical probe 200 can include a controller 109 which can be implemented entirely through analog electronic, or with a microcontroller and supporting circuits or with a DSP (Digital Signal Processing) or with an FPGA (Field-programmable gate array) or with ASIC (application-specific integrated circuit) or any variation or combination of these components.

The optical probe 200 can include an optical density sensor. The optical density sensor can be important in biological processes for measuring the growth rate of the culture. Embodiments can provide for on-line monitoring of the biomass density as well as the rate of culture growth in the reactor chamber. As the microorganisms/culture grow, the number of cells per unit volume increases over time. Light travelling through the culture aperture 108 is attenuated due to absorption and scattering. The amount of attenuation is dependent on the path length and the concentration of the absorbent particles/scattering centers. Thus, the number density of the cells can be measured consistently on the basis of attenuation of light as it travels through or reflected by the culture using a modulated light source and a tuned detector located at a fixed distance from the source with culture flowing in between or by a tuned detector located nearby the light emitter (to measure reflected light). The filtered and processed light intensity measured by the detector can then be used to give a measurement of the cell density of the culture. By measuring optical properties through the combination of absorption, fluorescence and scattered light techniques, the optical probe can have a wider dynamic range (measuring from the moment of inoculation up to higher culture densities).

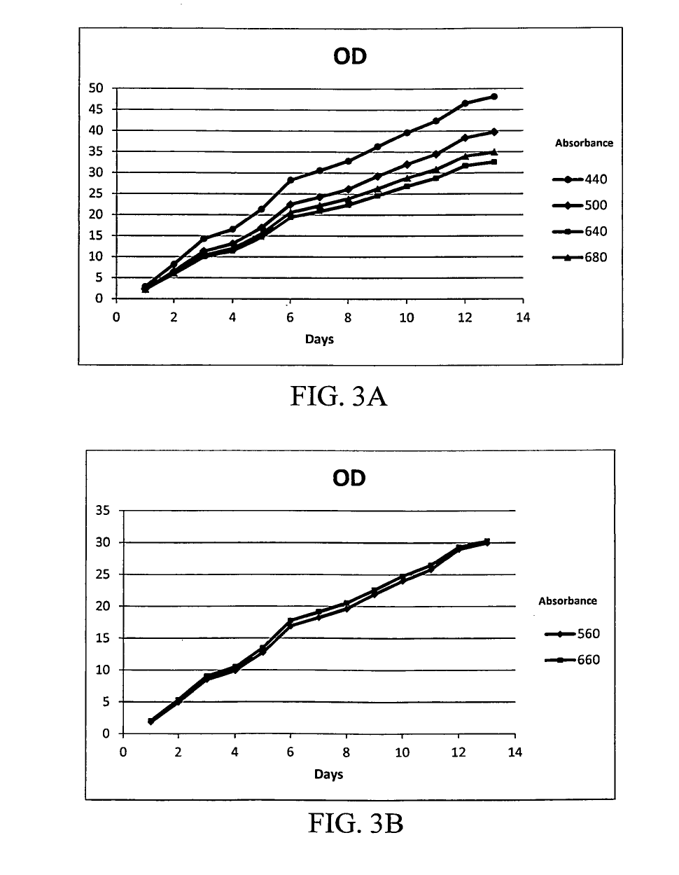

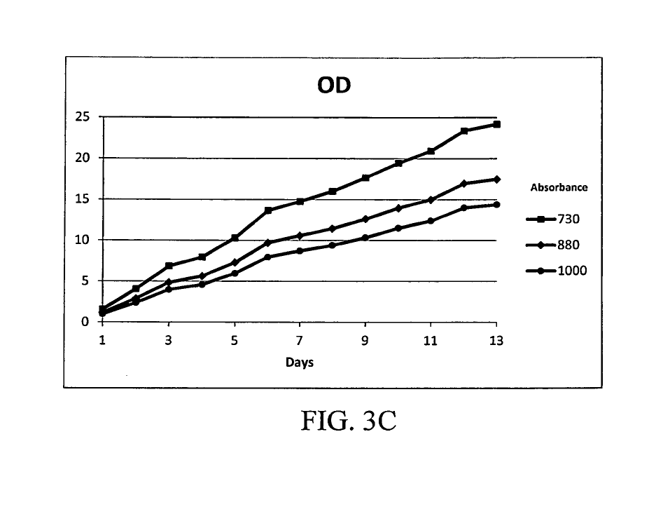

Turbidity (as it is also known in other fields to refer to optical density) is a measure of a liquid's lack of clarity and is an important indicator of any liquid quality. When a liquid has a high turbidity it is cloudy, while a low turbidity means the liquid is clear. The cloudiness is produced by light reflecting off particles in the liquid; therefore, the more particles in the liquid, the higher the turbidity. In the case of cell culture, higher turbidity is usually associated with more cells in the liquid. The selected wavelength for the optical density sensor can be anywhere above 700 nm, and for this implementation we are preferring 880 nm. A wavelength of 880 nm can allow accurate culture density measurement without complication by absorptions from photosynthetic pigments; however, other wavelength can be used without affecting advantages of the invention. FIG. 3A-C provides a plot of the optical density as the culture growth overtime. FIG. 3A is a plot of OD measurement using wavelengths 440 nm, 500 nm, 640 nm, and 680 nm. In FIG. 3B, the optical probe can measure the optical density of the microorganisms by emission and detection at specific wavelengths: 560 nm and 660 nm. FIG. 3C is a plot of the optical density of the microorganisms by emission and detection at specific wavelengths: 730 nm, 880 nm and 1000 nm.

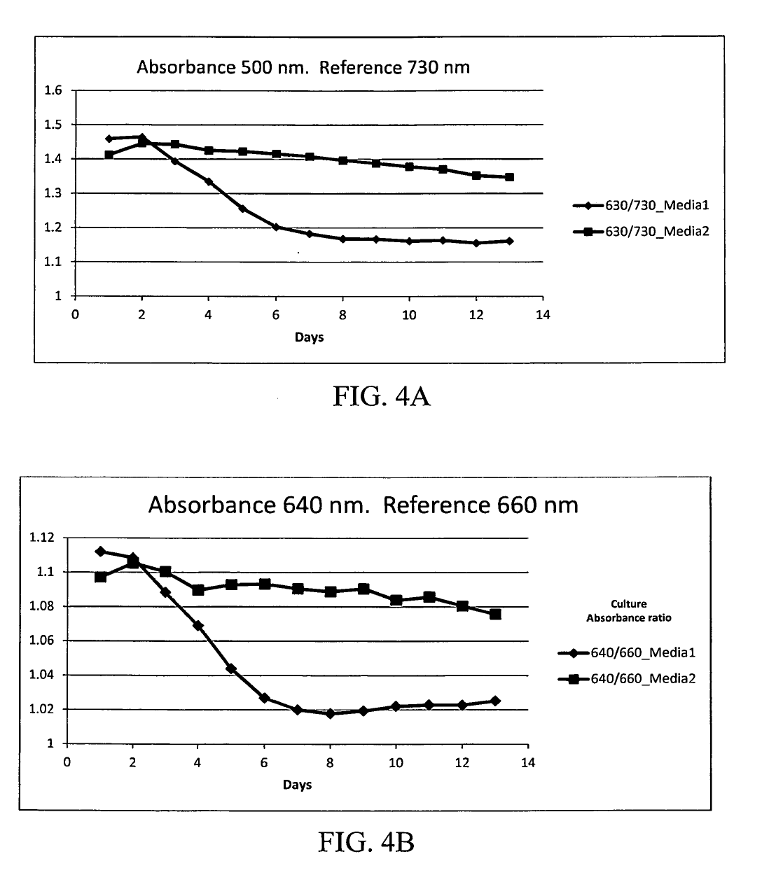

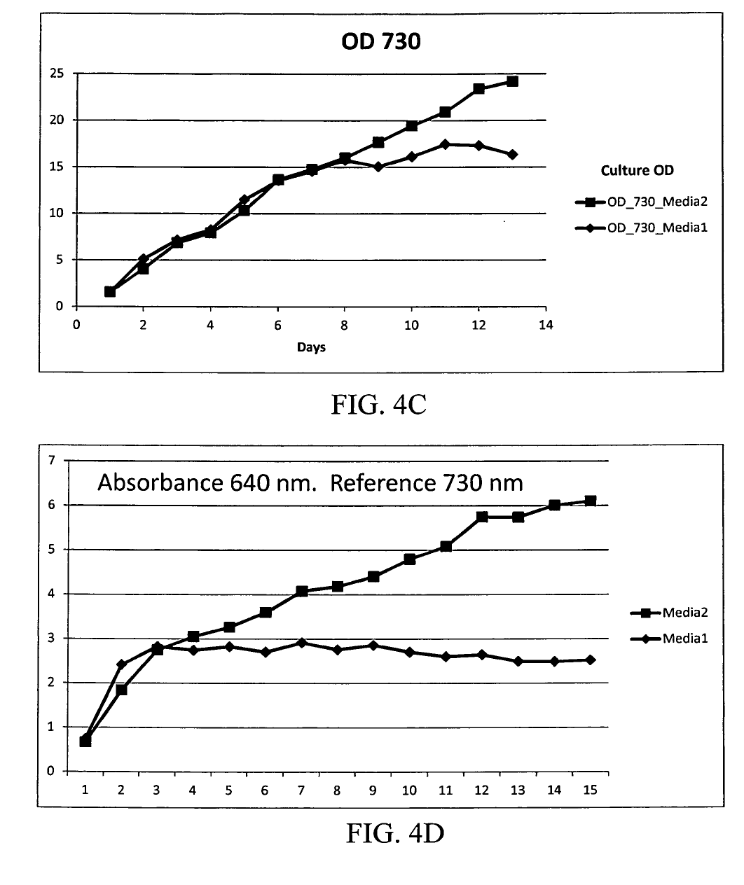

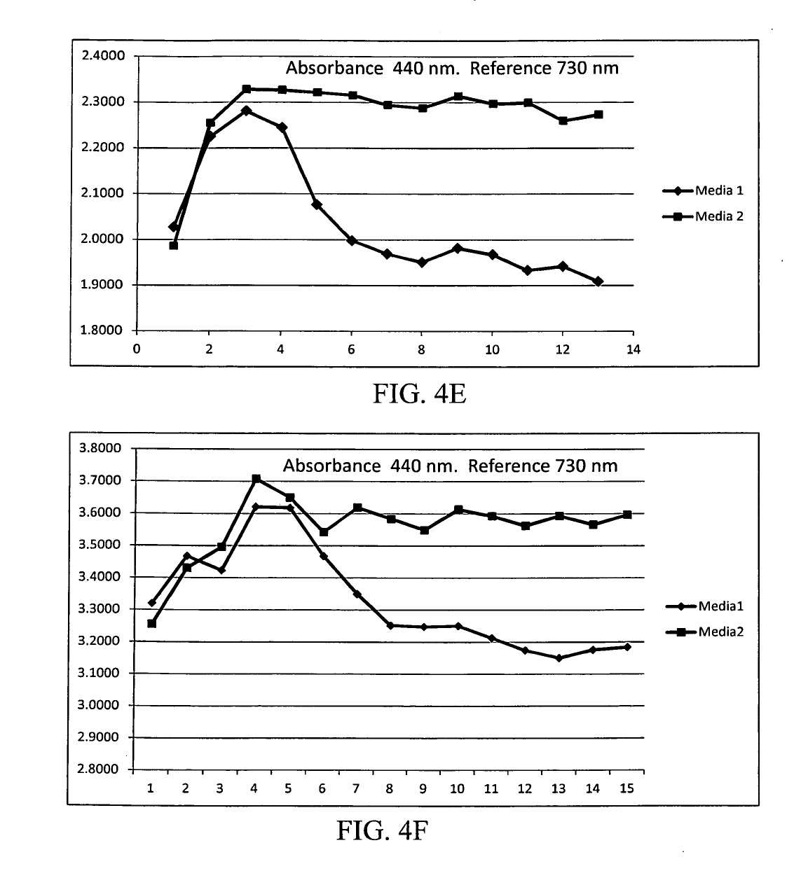

FIG. 4A is a plot of wavelength versus absorption ratios of the culture in accordance with an exemplary embodiment of the invention, based on the previously described operational wavelengths. A control culture (healthy) was run versus another one in limited nutrients media (sick) where signs of chlorosis will be manifested later on, the plots further demonstrate that embodiments can be used to differentiate both cultures. Two similar cultures were grown for several days: culture in Media 1 is more limited in nutrients and it can be seen that the method described in this patent can determine the culture degradation. Culture in Media 2 is also reaching a nutrient depletion later in the growth phase with signs of chlorosis. FIG. 4B is another metric from the same experiment, comparing a healthy control culture in Media 2 versus a nutrient depleted Media2 culture. FIG. 4C shows the optical density (OD) at 730 of both cultures showing the microorganisms growth under both conditions, and it is evident that the OD by itself cannot determine the organism's health condition, but it will see a decline in the OD increase farther in time. FIG. 4D shows a different metric (absorbance of 640 nm referenced to the absorbance at 730 nm) and a similar trend can be determine in determining the health of cultures (in the plot two cultures one in depleted media, versus another one in a rich media). FIG. 4E and FIG. 4F are graphs of wavelength versus absorption of the culture using the 440 nm referenced to 730 nm in accordance with an exemplary embodiment of the invention, there are early signs of chlorosis that can effectively trigger early intervention in the bioreactor process. A culture with early signs of chlorosis can be distinguished from the healthy culture one, providing opportunities for the process monitor and control system to take actions before any further organic deterioration can happen.

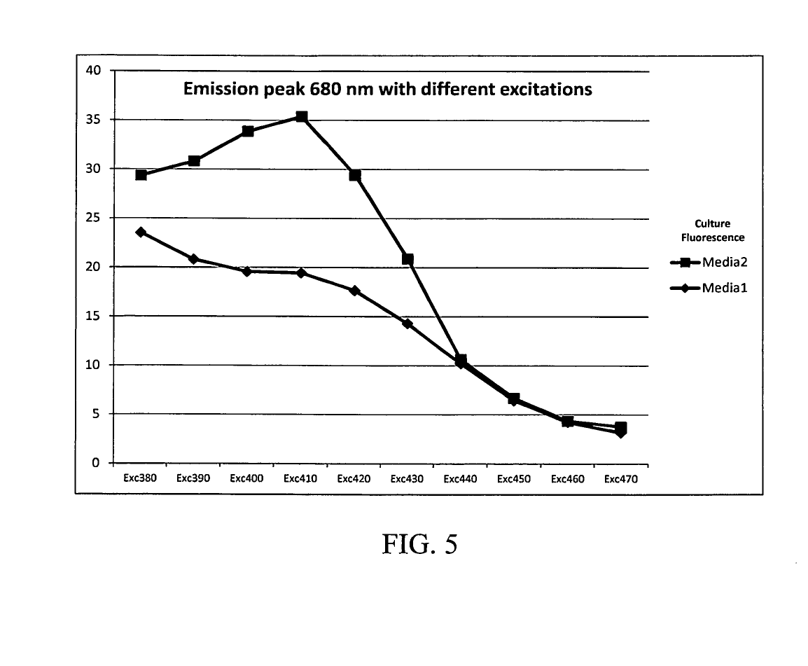

FIG. 5 is a plot of emissions of a healthy culture and a sick one at a varying excitation of 380 nm through 470 nm. A healthy culture has a gradual decrease in the emission at 680 nm; however, sick cultures (the ones with signs of chlorosis), has an increased emission when excited around 410 nm and then it declines as the excitation wavelength go down. The diminishing fluorescence response can be used as an early sign of a photosynthetic issues in the organic culture (i.e. chlorosis, etc.)

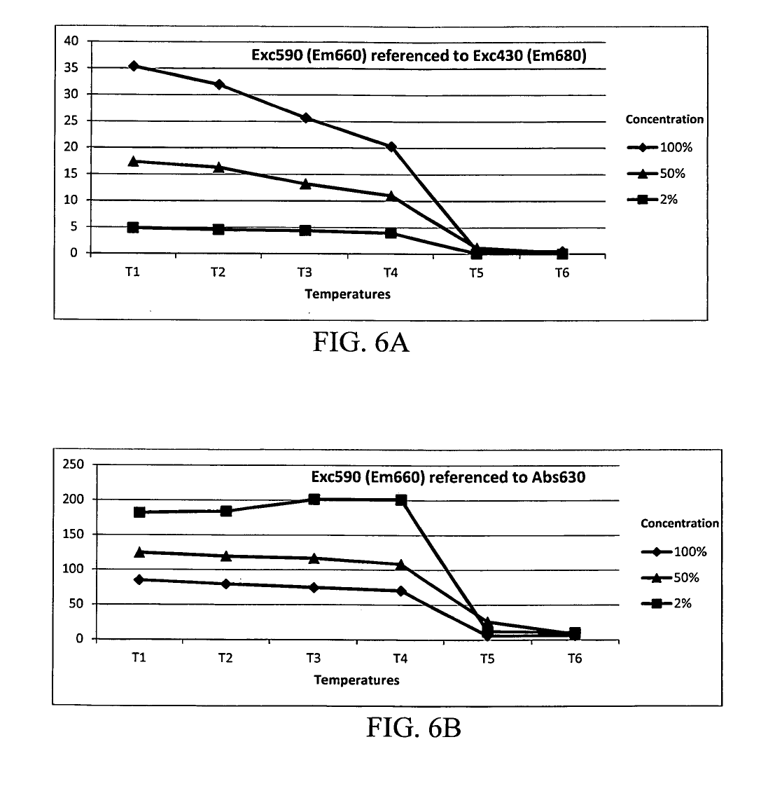

FIG. 6A is a plot of fluorescence emissions ratio of three healthy culture at different culture concentrations subjected to temperature increase; T5 is a fatal temperature for the culture and it can be seen from the plot that the method and apparatus proposed is able to determine that condition when the culture shows signs of chlorosis. FIG. 6B shows an alternative trend with the ratio of fluorescence and absorbance in accordance with the exemplary embodiment of the invention.

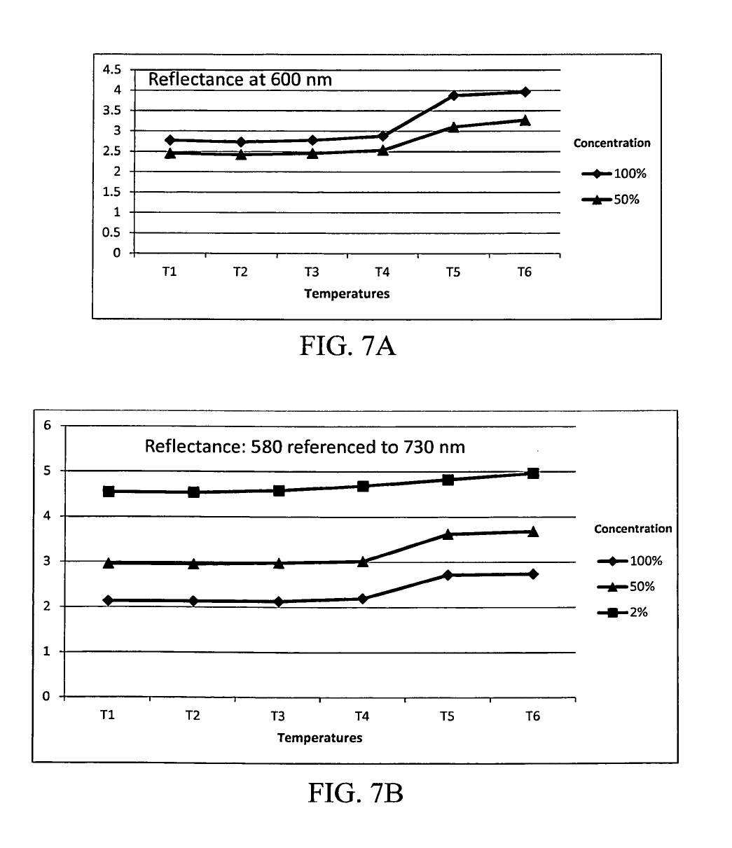

Looking at the culture color is an alternative way to determine culture healthiness. FIG. 7A shows the reflectance at 600 nm in accordance with the exemplary embodiment of the invention, for the same case described above (three healthy culture at different culture concentrations subjected to temperature increase). FIG. 7B shows a similar trend with the ratio of reflectance in accordance with the exemplary embodiment of the invention. Early signs of chlorosis are detected by exemplary embodiment of the invention.

The optical probe 200 can include a culture color sensor. The culture color can be important in biological processes for measuring health status of the culture and the growth rate of the culture. The hue and the tonality of the liquid culture are good indications of the organism's health status as it reflects the quality and quantity of photosynthetic pigments. An emitter and two detectors in, for example, in the green/yellow region can allow the measurement of the culture's “shade of green” and transition to an unhealthy state. The emitters and detectors can be anywhere in the region of 380 nm through 780 nm. Other colors can also be used since a culture in a “sick state” can be yellowish to brownish rather than green. Therefore, detectors in these regions might enable detection of the culture transitioning to other non-favorable states (not healthy, but rather getting sick). In the following table, data from different colors are shown (Green: 500 nm and 560 nm, Yellow: 580 nm and 590 nm, Orange: 600 nm) when a culture is subjected to increased temperatures; T4 is a critical temperature for the organisms.

TABLE 1
Microorganism culture under different
temperature stress and wavelength

| | 500 nm | 560 nm | 580 nm | 590 nm | 600 nm |
|---|---|---|---|---|---|
| T1 | 1.75 | 2.07 | 1.50 | 1.31 | 1.19 |
| T2 | 1.77 | 2.14 | 1.54 | 1.35 | 1.22 |
| T3 | 1.84 | 2.24 | 1.61 | 1.42 | 1.28 |
| T4 | 2.03 | 2.58 | 2.10 | 1.92 | 1.81 |
| T5 | 2.03 | 2.52 | 2.12 | 1.94 | 1.84 |

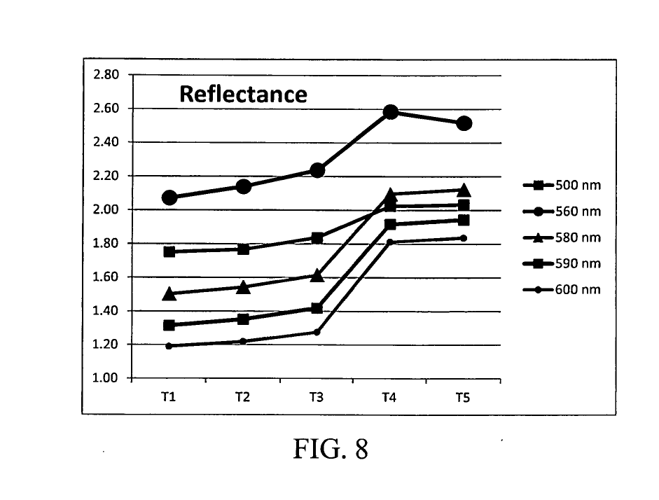

As can be seen from the Table 1 (or the plot in FIG. 8), the color at a particular wavelength will increase as the culture transition from a healthy state condition to a sick one (increasing temperature will affect seriously the health of culture, above a point where they are killed). This stress process can be similar to other conditions (nutrient depletion, photo inhibition, chlorosis, etc).

Therefore, information about the culture health can be obtained from detecting and analyzing the organism's color. The culture precise color shade can be important in biological processes for measuring health status of the culture and the growth rate of the culture.

The optical probe 200 can also include a photosynthetic pigments absorption sensor. Monitoring the photosynthetic pigments absorption peaks: 430 nm, 500 nm, 630 nm and 680 nm (the absorption peaks of an exemplary culture) provide significant information about the health of photosynthetic cultures. Embodiments can include tracking the absorption peaks over time and comparing them to wavelengths in the not photosynthetically active area as a reference in the same organism.

The optical probe 100 can include a fluorescence sensor. Fluorescence is the emission of light by a substance that has absorbed light radiation of a different wavelength. The fluorescence analysis technique allows noninvasive, near-instantaneous measurement of key aspects of photosynthetic light activity. The fluorescence method in combination with the previous techniques (turbidity and/or colorimetry) can provide some indication of the culture health status.

Photosynthetic pigments fluorescence can also be used to monitor photosynthetic activity and culture healthiness. An exemplary system for the fluorescence measurement method can include a probe wherein the temperature is controlled. Therefore, the organisms can be considered to be at a constant temperature. In another system, the temperature can be monitored and used as variables to correct or compensate the fluorescence measurements. In another exemplary system, the sensor can be located in a section that blocks external light (and/or the sensor enclosure can block any external light).

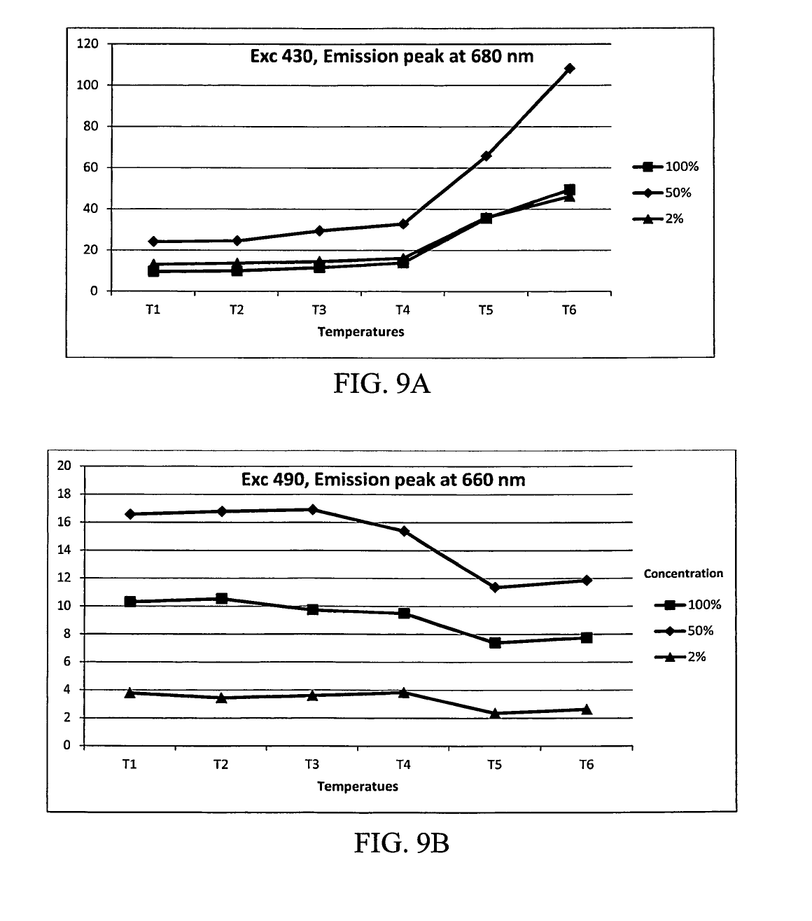

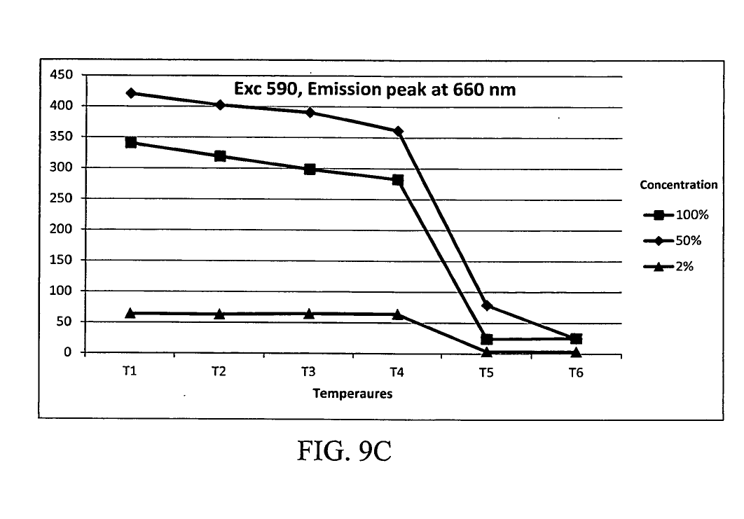

For phototrophic microorganisms that produce carbon based products of interest, a fluorescence with an excitation at 430 nm, the side-scattered emission light peak is at 630 nm as shown in FIG. 9A. A fluorescence with an excitation at 490 nm, the side-scattered emission light peak is at 660 nm as shown in FIG. 9B. A fluorescence with an excitation at 590 nm, the side-scattered emission light peak is at 660 nm as shown in FIG. 9C.

Although exemplary embodiments are described with reference to an optical probe, embodiments of the invention are not limited to a probe and can include a variety of probe configurations for culture growth devices and systems. Embodiments are not limited to one sensor system as shown in FIG. 1. Exemplary embodiments can utilize multiple apertures 208 and/or can comprise multiple sensors 202 in the same probe body 200. Embodiments are not limited to process pipes and can be incorporated into various tanks and or vessels for processing systems.

Signal Processing

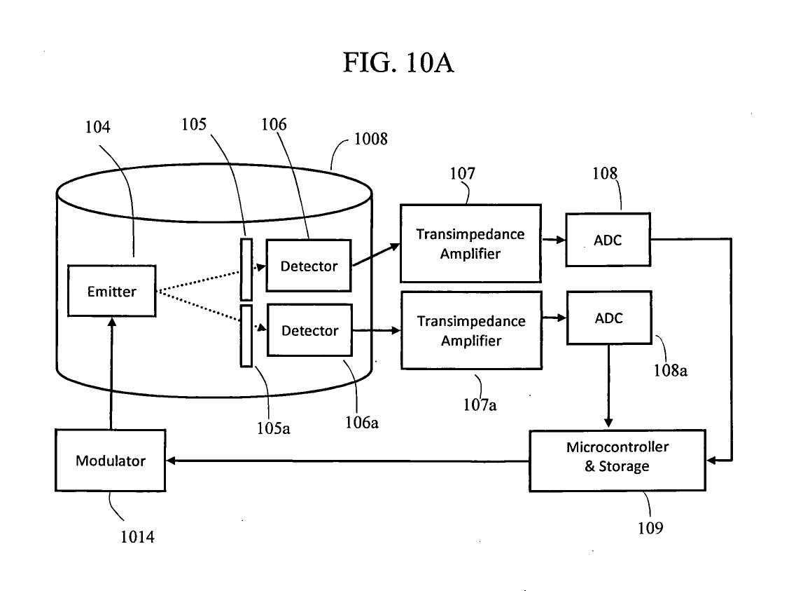

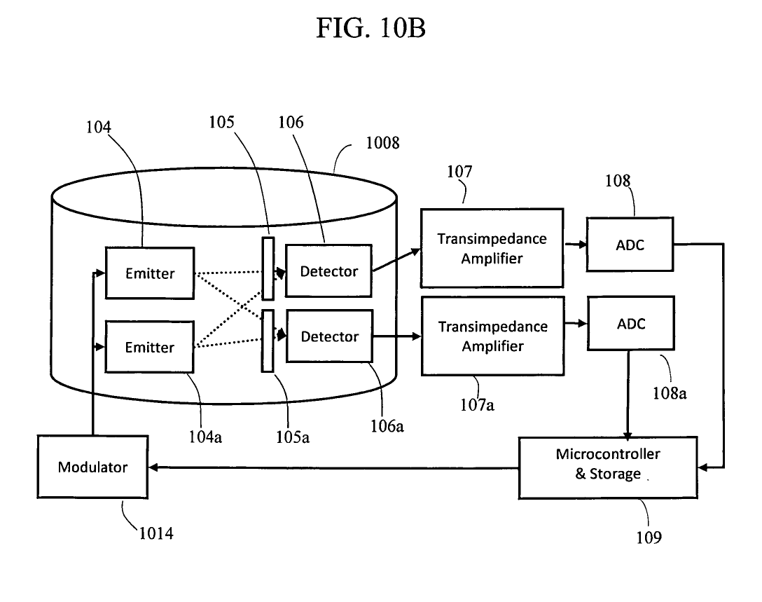

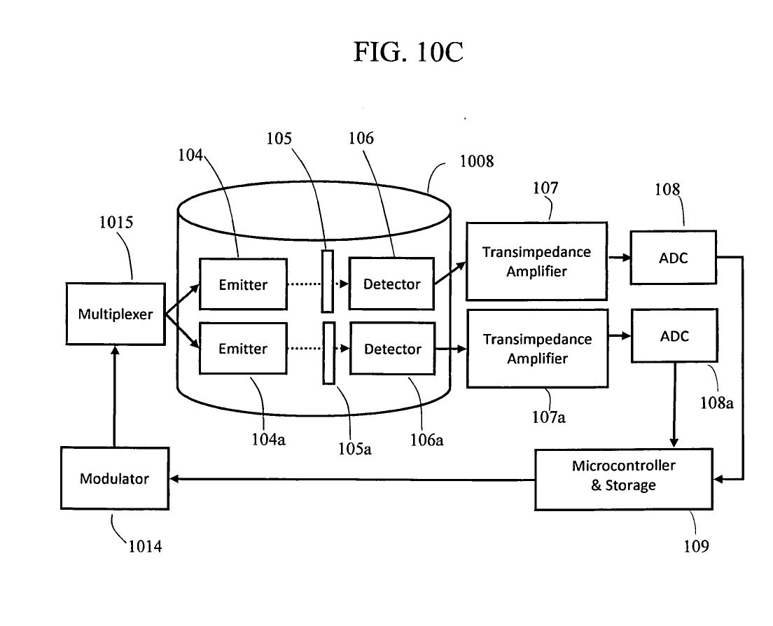

Referring to FIG. 10A, the system can include a microcontroller, microprocessor, digital signal processor (DSP, etc) or field programmable gate array (FPGA, ASIC, etc) 109 that can be performing the processing operations. The light emitted from the source emitter 104 is passed through the culture and or reflected by it, and is captured by the detector 106 and optionally by a secondary detector 106b in a different orientation (as shown in the set of FIG. 12). An optional optical filter 105 can be used to eliminate light from other wavelengths if necessary. Discrete components can be used for amplifying the signal from the emitter using 107 and performing the analog signal conversion using 108 received from the optical probe 100 or the probe sensor 102. The probe sensor 102 can include at least one emitter 104 and at least two detectors 106 and 106a within the optical probe 100 or the probe sensor 102, using an extra set of transimpedance amplifier 107a and ADC converter 108a, with an optional filter 105a inside the probe sensor 102. The microcontroller 109 can also communicate with a modulation 1014 to differentiate the light emitted by the emitter 104 from ambient light or other noises. Referring to FIG. 10B, additional emitters 104 and 104a can be included with additional detectors 106a with separate discrete amplifiers 107a and filters 105a, this additional emitter 104a and set of peripherals (105a, 106a, 107a and 108a) can be placed in different orientations (as shown in the set of FIG. 12) to maximize the reading of other optical features. Referring to FIG. 10C, optical probe can include multiple emitters 104 and 104a and multiple detectors 106 and 106a, which are controlled by a multiplexer 1015 1015C. Multiple light emitters and/or detectors can be used to improve the wavelength coverage, and/or light scattered/reflected features.

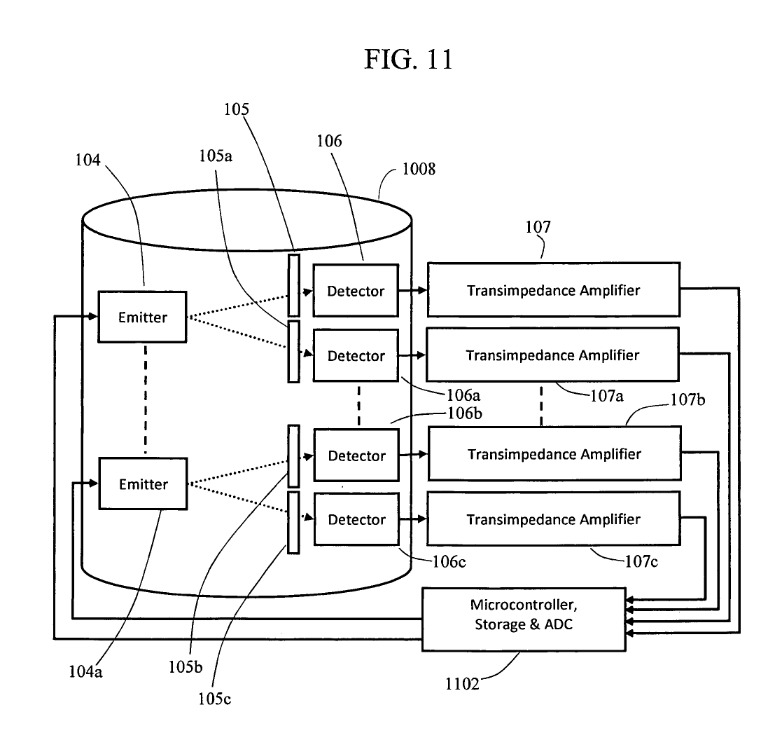

Referring to FIG. 11, embodiments can also include an integrated system by integrating various components into the microcontroller 1102. This can be accomplished because the microcontroller unit 1102 can have a section performing specific functions (ADCs, DACs, etc) and or because they are implemented in firmware (filtering, Modulation, PWM, multiplexing, etc). A similar optical probe within the flow microorganisms and culture medium 1008 can include one or more emitters 104, 104a, etc and multiple detectors 106, 106a, 106b, 106c, etc with various filters 105, 105a, 105b, 105c, etc and amplifiers 107, 107a, 107b, 107c, etc. The microcontroller 1102 can be used process the signals emitted and detected by the overall integrated system. The additional emitters and receivers can be organized in different configurations (as shown in the set of FIG. 12) to maximize the reading of other optical characteristics.

Optionally, the microcontroller 109 or 1102 (processor, DSP, FPGA, or any processing element in the system) might also need to read the culture temperature and other variables. The temperature sensor can be a thermocouple, RTD or thermistor. The temperature sensor can be as close as possible to the process and the sensors as to determine the sensor operating temperature without making contact with the flow of microorganisms and culture medium.

Multiple configuration of emitter 104 and set of receivers 106 can be used to exercise the photosynthetic organisms optical response as shown in the configurations of FIG. 12A through FIG. 12E. Cost, accuracy as well as target function of the invention implementation are factors that weight in the selection of one of all the configurations with simple approach or a combination of methods.

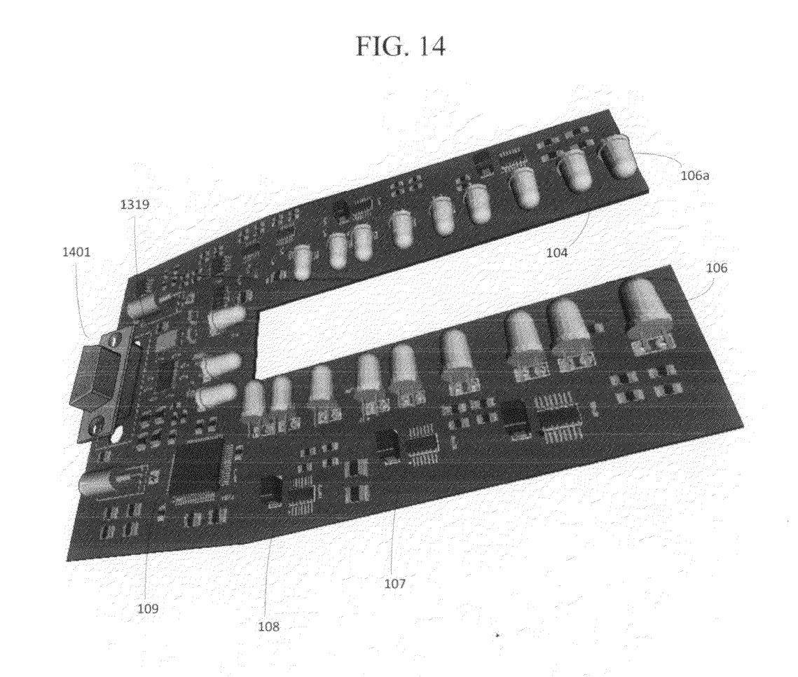

FIG. 14 shows a 3D view of one of the potential implementations of the optical sensor model described in this invention with multiple sets of emitters 104 and receivers 106, 106a, etc. This is a potential PCB implementation of the many alternatives to deploy the concepts in accordance with an exemplary embodiment of the invention. The microcontroller 109 will get the ADC data 108 from the transimpedance amplifiers 107. The switch 1319 allows the sensor to activate a process element in the final application, while the communication port 1401 allows an interface to network with industrial processors, including PLCs, DCSs or any other computerized system.

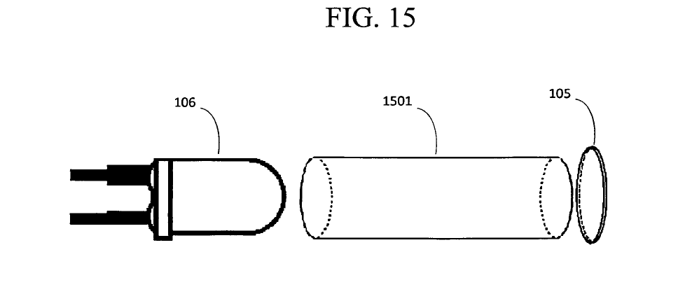

FIG. 15 shows a detailed view of the receiver 106 with an additional light guide element 1501 and the optional filter 105. Optional support element of light blocking attachments might be added for better optical reception, mounting and support. Additional attachments can be used to simplify manufacturing and operation.

A variable gain amplifier can be employed as the equivalent to 107 to automatically adjust the receiver 106 signal gain in real-time. The gain profile may be scheduled based upon information from the sun's light intensity, time of day, or updated automatically based upon the magnitude of the received signal. Such a feature enables measurement of specific characteristics that are sensitive to active photosynthesis, without saturating the analog-to-digital circuitry. Similarly, the light source intensity may be varied to further optimize the measurement dynamic range and to better observe weak spectral features.

Algorithm and Signal Processing

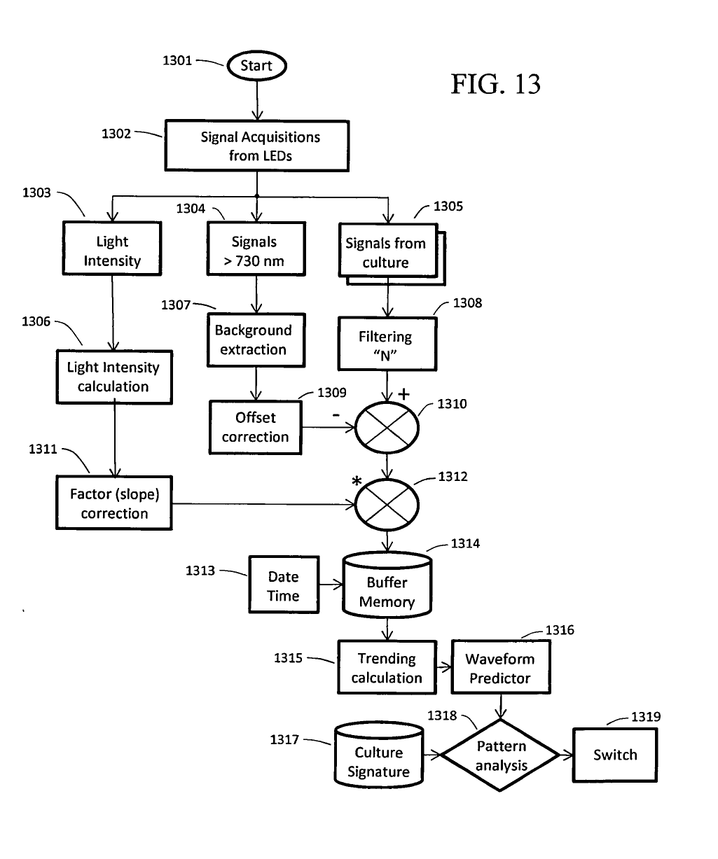

Signal processing is required to achieve better measurements. A source of measurement instability is background instabilities of the culture spectrum, which may be due to slight optical alignment changes (for example due to temperature variations), emitter 104 degradation, dirty filter optics, vibration from the process, etc. The algorithm and signal processing FIG. 13 is employed to ensure long-term measurement signal stability and measurement accuracy.

Measured signals from the detectors 106 (or 106a, 106b, and or 106c) are acquired and processed by the microcontroller and storage 109, multiple signals from the culture 1305 can be expected in this algorithm but also a single channel is possible. Signals from the culture as well as the reference are either absorbance, reflectance or fluorescence. To further improve wavelength stability/repeatability, the measured signal just before the A/D conversion is over-sampled, i.e. the signal is digitized at a frequency significantly higher than the Nyquist criterion which is required to accurately reproduce the analog signal digitally. Such over-sampling is achieved by employing higher sampling frequency set forth by the microcontroller 109, and then filtered through 1308.

A reference signal 1304 with wavelength preferably above 730 nm (although reference signals at other wavelengths are also possible) provide the background information 1307 required to normalize signals and remove variations due to normal growth, suspended particles, dirtiness, etc. This compensation allows to remove the offset 1309 which help to maximize the signal over a wider range of operation. A spectral differentiation 1310 is used to remove the background variations. The spectral differentiation algorithm is of the form S_new(n)=S(n)−Sb(n), or variations thereof, where S_new is the resulting baseline-corrected spectrum, S is the original spectrum, Sb is the background extracted from the processes 1304, 1307 and 1309, and n is the data element of the spectrum.

Light intensity variations from the emitter 104 or from other sources in the process (sun light on culture, etc.) can create some variability on the spectrum signal from the culture. Therefore measuring the light intensity (through 106c or variations on 106b and or variations on 106a) can help to determine a factor to account for light intensity calculation 1306 which can be applied as 1311 to correct the slope the measured signal from the culture through 1312. The spectral correction algorithm is of the form S_new(n)=S(n)*Ss(n), or variations thereof, where S_new is the resulting slope-corrected spectrum, S is the original spectrum (whether corrected for background in 1310 or not), Ss is the factor associated to light intensity variations measured and calculated through 1303, 1306 and 1311, and n is the data element of the spectrum.

In another embodiment, the spectra is time stamped 1313 and stored in a database 1314 to follow the evolution of the culture over time. Based on additional factors (temperature, time, etc) a trending evolution 1315 is possible and the trajectory of growth (waveform prediction) 1316 can be established. Particularities of the photosynthetic culture can be stored as a signature 1317 which can be used to determine the correct growth based on current data through a pattern analysis 1318. This information together with user set points entered previously in the system will allow the triggering of a switch 1319 to activate an alarm, or activate the release of compounds required to mitigate a deficiency in the culture, process or system.

Optical Probe Biomass Productivity

The optical probe and system can be incorporated into a solar biofactory or photobioreactor and also provide methods to achieve organism productivity as measured by production of desired products, which includes cells themselves.

The optical probe allows microorganism's processes to be automated by measuring key parameters associated to the culture health and efficient productivity. An healthy organism will be effective and efficient in producing the by-product of interest.

### DEFINITIONS

Suitable phototrophic microorganisms can produce the target by-product and/or the phototrophic microorganism itself can be processed as feedstock for the production of a desired by-product. Particularly suitable phototrophic microorganisms can be natural organism as cyanobacteria or genetically engineered photosynthetic organisms

Typical by-products from the microorganisms can be amino acids, antibiotics, vitamins, nutrients, food, fuels, biofuels, biomass, medications, chemicals or other substances.

As used herein, “light of a wavelength that is photosynthetically active in the phototrophic microorganism” refers to light that can be utilized by the microorganism to grow and/or produce by-products of interest, for example: amino acids, antibiotics, vitamins, nutrients, food, fuels, biofuels, biomass, medications or other substances.

As used herein, “transparent” refers to an optical property that allows passage of light of a wavelength that is photosynthetically active in the phototrophic microorganism and or other desirable wavelengths of light.

“Phototrophs” or “photoautotrophs” are organisms that carry out photosynthesis such as, eukaryotic plants, algae, protists and prokaryotic cyanobacteria, green-sulfur bacteria, green non-sulfur bacteria, purple sulfur bacteria, and purple non-sulfur bacteria. Phototrophs include natural and engineered organisms that carry out Photosynthesis and hyperlight capturing organisms.

The optical probe of the present invention are adapted to support a biologically active environment that allows chemical processes involving photosynthesis in organisms such as phototrophic organisms to be carried out, or biochemically active substances to be derived from such organisms.

As used herein, “organisms” or “microorganisms” encompasses autotrophs, phototrophs, heterotrophs, engineered light capturing organisms and at the cellular level, e.g., unicellular and multicellular.

A “spectrum of electromagnetic radiation” as used herein, refers to electromagnetic radiation of a plurality of wavelengths, typically including wavelengths in the infrared, visible and/or ultraviolet light. The electromagnetic radiation spectrum is provided by an electromagnetic radiation source that provides suitable energy within the ultraviolet, visible, and infrared, typically, the sun.

As used herein, “light” generally refers to sunlight but can be solar or from artificial sources including incandescent lights, LEDs, fiber optics, metal halide, neon, halogen and fluorescent lights.

As used herein, the “optical density” is measured through spectral characteristic of the culture.

Throughout this specification and claims, the word “comprise” or variations such as “comprises” or “comprising,” will be understood to imply the inclusion of a stated integer or group of integers but not the exclusion of any other integer or group of integers.

The foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of this invention are presented for purposes of illustration and description. They are not intended to be exhaustive or to limit the invention to the precise forms disclosed; obviously many modifications and variations are possible in view of the above teachings. The embodiments were chosen and described in order to best explain the principles of the invention and its practical applications. These procedures will enable others, skilled in the art, to best utilize the invention and various embodiments with various modifications. It is intended that the scope of the invention be defined by the following claims and their equivalents. Modifications and substitutions by one of ordinary skill in the art are considered to be within the scope of the present invention, which is not to be limited except by the following claims.

While this invention has been particularly shown and described with references to example embodiments thereof, it will be understood by those skilled in the art that various changes in form and details may be made therein (for example non photosynthetic organisms, etc) without departing from the scope of the invention encompassed by the appended claims. The scope of protection of the invention is not limited to the examples given hereinabove. The invention is embodied in each novel characteristic and each combination of characteristics, which particularly includes every combination of any features which are stated in the claims, even if this feature or this combination of features is not explicitly stated in the claims or in the examples.

The invention claimed is:

1. An optical probe apparatus comprising:
An immersive, optical probe positioned at least in part within the process chamber and having a (1) sample volume open to phototrophic microorganisms and culture medium of the reactor chamber and (2) including at least one emitter adapted for controlled intensity variation of emitted light and at least two detectors adapted for detection of light, wherein the at least one emitter and a first of the at least two detectors are positioned such that a focused beam of light emitted from the emitter passes along a line through the sample volume to be received by a light receiving area of the first detector, and a second of the at least two detectors which is positioned such that its light receiving area is oriented towards the focused beam of light and at certain degree angle from the line (following the Law of Reflection); and
An enclosure for the optical sensor and the electronics in a shape and geometry that allows for maximum fluid sensing without limiting the process itself;
A series of light emitters and detectors that excite the photosynthetic pigments of photosynthetic microorganisms.

2. An optical probe of claim 1, wherein the at least one emitter and the at least two detectors are modulated.

3. An optical probe of claim 1, wherein the at least one emitter are at wavelengths greater than 360 nm and below 1,000 nm.

4. An optical probe of claim 1, wherein the optical probe is adapted to measure an optical density of the microorganisms and culture medium by evaluating (1) scattered light originating from the sample volume and measured by the second detector with (2) transmitted light measured by the first detector.

5. An optical probe of claim 1, wherein the optical probe has an emitter adapted for emission of light at a wavelength range corresponding to at least one photosynthetic pigment of the phototrophic microorganism, and the emitter, sample volume, and first and second detector are positioned for optical density measurements.

6. An optical probe apparatus comprising:
An immersive, continuous operating optical probe including at least one modulated emitter and at least two modulated detector wherein the optical probe measures an optical density of the microorganisms and the color of reflected light off the microorganisms; and
An enclosure for the optical sensor and the electronics in a shape and geometry that allows for maximum fluid sensing without limiting the process itself.

7. An optical probe of any one of claim 1 or 6, wherein the optical probe measures a visible color of reflected light off the microorganisms at wavelengths of about 380-780 nm.

8. An optical probe of any one of claim 1 or 6, further comprising a microcontroller and storage wherein the optical probe measures a color of reflected light off the microorganisms by emission and detection at a wavelengths of about 500 nm, 560 nm, 580 nm, 590 nm, 600 nm, and 630 nm and the microcontroller compares past color measurements of the optical probe with current color measurements and determines a health condition status of the microorganisms.

9. An optical probe of any one of claim 1 or 6, wherein the optical probe measures a photosynthetic response of the microorganisms by emission and detection at a wavelength of ranges of 400 nm to 460 nm, 480 nm to 540 nm, 620 nm to 680 nm.

10. An optical probe comprising:
An immersive, continuous operating optical probe including at least one modulated emitter and at least two modulated detector wherein the optical probe measures an optical density of the microorganisms and a photosynthetic efficiency of the microorganisms by measuring a fluorescence of the microorganisms; and
An enclosure for the optical sensor and the electronics in a shape and geometry that allows for maximum fluid sensing without limiting the process itself.

11. An optical probe of any one of claim 1 or 10, wherein the optical probe measures a photosynthetic activity of the microorganisms by measuring the fluorescence of the microorganisms.

12. An optical probe of any one of claim 1 or 10, wherein the optical probe measures a fluorescence activity of the microorganisms by using an excitation at a wavelength range of 380 nm to 420 nm and detection at a wavelength of about 680 nm.

13. An optical probe of any one of claim 1 or 10, wherein the optical probe measures a fluorescence activity of the microorganisms by using an excitation at a wavelength of 490 nm and detection at a wavelength of about 660 nm.

14. An optical probe of any one of claim 1 or 10, where in the optical probe measures absorption and scattered light to widen dynamic range and accurately measure culture optical density from the moment of inoculation up to higher culture densities.

15. An optical probe of any one of claim 1 or 10, wherein the optical probe is adapted to measure an optical density of the microorganisms and culture medium by evaluating (1) scattered light originating from the sample volume and measured by the second detector with (2) transmitted light measured by the first detector.

16. An optical probe comprising:
An immersive, continuous operating optical probe including at least one modulated emitter and at least two modulated detector wherein the optical probe measures an optical density of the microorganisms; a color of reflected light off the microorganisms; and photosynthetic activity of the microorganisms by measuring a fluorescence of the microorganisms; and
An enclosure for the optical sensor and the electronics in a shape and geometry that allows for maximum fluid sensing without limiting the process itself.

17. A method for determining the quality and healthiness condition of phototrophic microorganisms in the optical probe, the method comprising:
measuring optical density of microorganisms and culture medium therefor in the sample volume of the optical probe,
flowing the phototrophic microorganisms and culture medium therefor through the optical path;
determining a growth rate from several optical density measurements over time performed by the optical probe, and
measuring different wavelength response of the microorganism culture in the photosynthetically active region.

18. The method of claim 17, further comprising measuring transmission and/or absorbance associated with a photosynthetic pigment of the microorganisms and culture medium therefor in the sample volume of the optical probe at suitable emission and detection wavelength to determine a color of the microorganisms and culture medium therefor, and determining a health status of the microorganisms by (1) evaluating several transmission and/or absorbance measurements performed by the optical probe over time, and/or (2) evaluating at least one transmission and/or absorbance measurement performed by the optical probe in view of previously established information corresponding to health status of the microorganisms.

19. The method of claim 17, further comprising analyzing fluorescence of the microorganisms in the sample volume of the optical probe at suitable excitation and detection wavelength, and determining culture healthiness condition of the microorganisms by (1) evaluating several transmission and/or absorbance measurements performed by the optical probe over time, and/or (2) evaluating at least one transmission and/or absorbance measurement performed by the optical probe in view of previously established information corresponding to health status of the microorganisms.

20. The method of claim 17, wherein the process is adapted to analyze absorbance, reflected and fluorescence light off the photosynthetic microorganisms overtime.

21. An optical probe system comprising:
An optical probe position inside the flow of a photosynthetic microorganism process, with a multitude of emitters and receivers in the photosynthetic sensible to light of a wavelength that is photosynthetically active in the phototrophic microorganism,
Emitters and receivers in the NIR region arranged to measure absorbance and reflectance of light,
Adequate light filters to allow the detection of specific wavelengths according to the feature to be excited,
A method that enable the detection of microorganisms degradation by monitoring performance features on the photosynthetic pigment,
A method that track multiple photosynthetic features and their performance overtime in relation to optical density in the non-photosynthetic active region.

22. An optical probe system of claim 21, where in the system compares the photosynthetic response against a set of signatures corresponding to the organisms of interest.

23. An optical probe system of claim 21, where a contact is activated based on the sensor information to actuate on another local subsystem.
